(19) United States
(12) Patent Application Publication
(10) Pub. No.: US 2005/0246788 A1
(43) Pub. Date: Nov. 3, 2005

(54) Self-incompatible transgenic plants

(76) Inventor: Shihshieh Huang, Stonington, CT (US)

Correspondence Address: Monsanto CO, 62 Maritime Dr, Mystic, CT, 06355, US

(21) Appl. No.: 11/100,097

(22) Filed: Apr. 5, 2005

Related U.S. Application Data
(60) Provisional application No. 60/563,793, filed on Apr. 20, 2004.

(51) Int. Cl. A01H 1/00; C12N 15/82; A01H 5/00

(52) U.S. Cl. 800/278; 435/468; 800/320.1; 800/320.2; 800/320.3; 800/312; 800/306; 800/314

(57) ABSTRACT

Self-incompatible, transgenic lines of naturally inbreeding-capable crops can be cross pollinated to produce hybrid seed with copies of allelic DNA from each parent. Each transgenic line comprising in its genome a segment of exogenous, allelic DNA from an S-locus of a self-incompatible plant. Each transgenic line produces pollen which is incapable of fertilizing lines with said the same allelic DNA from an S-locus that is present both the paternal and maternal lines.

### RELATED APPLICATION

This application claims priority to provisional application Ser. No. 60/563,793 filed Apr. 20, 2004, incorporated herein by reference.

### INCORPORATION OF SEQUENCE LISTING

The following sequence listing is identical to the sequence listing submitted in provisional application No. 60/563,793 where the computer readable form was in the file named “53637.ST25.txt” which is 11 kilobytes (measured in MS Windows) and which was created on Apr. 20, 2004 on compact disc and is incorporated herein by reference.

### FIELD OF THE INVENTION

Disclosed herein are self-incompatible transgenic plants, i.e. plants that are incapable of self fertilization, and methods of making and using such plants.

### BACKGROUND

In pollination of flowering plants DNA from male tissue pollen merges with DNA in a female tissue ovary after pollen in contact with a stigma initiates pollen tube growth in a pistil to an ovary. The cells in the pollen tube carry “male” DNA from the pollen; the pistil itself carries “female” DNA of the host plant. In plants capable of self-fertilization, pollen from the host plant generates a pollen tubes which run uninhibited to ovaries. In plants with gametophytic self-incompatibility, pollen tubes initiated from host plant pollen are inhibited from reaching ovaries.

In U.S. Pat. No. 5,585,543 Kao disclosed that suppression of S-locus RNase was effective in making petunia self-pollinating. Recently, Sijacic et al. (submitted to Nature) disclosed that the S-locus in Petunia inflata contains allelic RNase gene with a female-specific promoter and an allelic S-locus F-box gene (SLF) with a male-specific promoter. In Petunia inflata and other naturally self incompatible plants allelic SLF is produced in male specific tissue, e.g. in pollen tubes, and allelic RNase is produced in female tissue, e.g. in pistil.

When pollen tubes are initiated by pollen from a host plant with an allelic S-locus, the allelic pollen-sourced SLF protein in the pollen tubes does not inhibit the activity of the RNase in pistil tissue. The result is inhibition of continued pollen tube growth, effecting self-incompatibility.

When pollen tubes are initiated from pollen with allelic S-locus genes that are distinct from the allelic S-locus genes of the host plant, the allelic SLF inhibits RNase activity allowing continued growth of such pollen tubes.

The identification of multiple S-loci allows pollination between plants having different alleles of S-locus genes.

### SUMMARY OF THE INVENTION

This invention provides self-incompatibility to transgenic plants that are naturally capable of inbreeding. Self-incompatibility is effected by creating transgenic plant lines with allelic S-locus genes, e.g. from a naturally self-incompatible plant such as Petunia inflata. Thus, the benefits of hybrid crops can be more effectively achieved in plants that are natively self-pollinating when self-incompatibility is introduced into the plant. For instance, the production of hybrid corn seed could be improved via crosses of two-self-incompatible corn lines which do not require mechanical or chemical detasseling. Moreover, hybridization can be more readily achieved in naturally-inbreeding-capable crops such as rice, wheat, canola, cotton, soybean and the like. Preferred crop plants also contain recombinant DNA imparting herbicide resistance and/or pest resistance.

More specifically, this invention provides a self-incompatible, transgenic line of a naturally inbreeding-capable crop. Such self-incompatible transgenic plant comprises in its genome recombinant DNA comprising a segment of exogenous, allelic DNA from an S-locus of a self-incompatible plant. Such recombinant DNA comprises the allelic DNA encoding an allelic RNase and allelic DNA encoding the cognate allelic SLF protein. The DNA can comprise the native promoters of the RNase and SLF genes; alternatively, the DNA can comprise other promoters, e.g. promoters from the host plant.

With multiple transgenic lines of a crop with different allelic S-locus genes, improvement to methods for producing hybrid plants can be achieved. Thus, this method provides a method of producing hybrid plants of an inbreeding-capable crop. The method comprises:
(a) introducing into the genome of a first line of a crop plant a segment of allelic DNA from an S-locus of a self-incompatible plant to provide a self-incompatible, transgenic line of said crop plant,
(b) allowing female tissue of a second line of the crop to be fertilized by pollen from the self-incompatible, transgenic line of said crop, wherein the second line of the crop does not have the allelic S-locus DNA that is said self-incompatible, transgenic line of said crop.
   More specifically, the second line of the crop can be wild type, i.e. without any alleleic S-locus DNA, or the second line of the crop can be a self-incompatible, transgenic line of the crop but with different allelic S-locus DNA from the allelic S-locus DNA that is in the first line of the crop. The method for producing hybrid plants is enhanced when both crop lines are transgenic with unique allelic S-locus DNA in each line.

Although hybrid corn is well known, because corn can self pollinate, hybridization requires intervention to prevent self pollination. This invention provides a method of increasing the yield of hybrid corn seed comprising planting adjacent corn plants of self-incompatible, transgenic lines of corn, wherein each of the transgenic corn lines has in its genome a unique segment of exogenous, allelic DNA from an S-locus of a self-incompatible plant. That is, each corn line has a different allele of allelic S-locus DNA.

The methods of this invention can also be applied to increasing yield in a self-pollinating plants by growing hybrid seed from transgenic crops of this invention, e.g. crops such as rice, wheat, canola, soybean and cotton. The method comprises growing a crop from hybrid seed produced from cross fertilization of two transgenic plants of the crop where of the two plants has in its genome exogenous DNA from at least one S-locus from a self-incompatible plant.

This invention also provides hybrid seed comprising at least two allelic copies of DNA coding for S-locus RNase and two allelic copies of DNA coding for S-locus F-box protein.

### DETAILED DESCRIPTION OF PREFERRED EMBODIMENTS

SEQ ID NO:1 is a segment of allelic genomic DNA from the S-locus of Petunia inflata for the RNase gene, where the RNase coding sequence exons are nucleotide 2027-2260 and 2367-2997.

SEQ ID NO:2 is a segment of allelic genomic DNA from the S-locus of Petunia inflata for the SLF gene, where the SLF coding sequence is 2068-3237.

Crop plants of interest in the present invention include, but are not limited to, soybean (including the variety known as Glycine max), cotton, canola (also known as rape), corn (also known as maize and Zea mays), wheat, sunflower, sorghum, alfalfa, barley, millet, rice, fruit and vegetable crops.

As used herein an “herbicide resistance” trait is a characteristic of a transgenic plant that is resistant to dosages of an herbicide that is typically lethal to a progenitor plant. Such herbicide resistance can arise from a natural mutation or more typically from incorporation of recombinant DNA that confers herbicide resistance. Herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and gluphosinate herbicides. To illustrate the that production of transgenic plants with herbicide resistance is a capability of those of ordinary skill in the art reference is made to U.S. patent application publications 2003/0106096A1 and 2002/0112260A1 and U.S. Pat. Nos. 5,034,322; 6,107,549 and 6,376,754, all of which are incorporated herein by reference.

As used herein an “pest resistance” trait is a characteristic of a transgenic plant is resistant to attack from a plant pest such as a virus, a nematode, a larval insect or an adult insect that typically is capable of inflicting crop yield loss in a progenitor plant. Such pest resistance can arise from a natural mutation or more typically from incorporation of recombinant DNA that confers pest resistance. For insect resistance, such recombinant DNA can, for example, encode an insect lethal protein such as a delta endotoxin of Bacillus thuringiensis bacteria or be transcribed to a dsRNA targeted for suppression of an essential gene in the insect. To illustrate that the production of transgenic plants with pest resistance is a capability of those of ordinary skill in the art reference is made to U.S. Pat. Nos. 5,250,515 and 5,880,275 which disclose plants expressing an endotoxin of Bacillus thuringiensis bacteria, to U.S. Pat. No. 6,506,599 which discloses control of invertebrates which feed on transgenic plants which express dsRNA for suppressing a target gene in the invertebrate, to U.S. Pat. No. 5,986,175 which discloses the control of viral pests by transgenic plants which express viral replicase, and to U.S. Patent Application Publication 2003/0150017 A1 which discloses control of pests by a transgenic plant which express a dsRNA targeted to suppressing a gene in the pest, all of which are incorporated herein by reference.

The present invention contemplates the use of DNA for imparting self-incompatibility in plants, e.g. DNA expressing S-locus RNase and SLF. Such DNA is assembled in recombinant DNA constructs using methods known to those of ordinary skill in the art. A useful technology for building DNA constructs and vectors for transformation is the GATEWAY™ cloning technology (available from Invitrogen Life Technologies, Carlsbad, Calif.) uses the site specific recombinase LR cloning reaction of the Integrase/att system from bacterophage lambda vector construction, instead of restriction endonucleases and ligases. The LR cloning reaction is disclosed in U.S. Pat. Nos. 5,888,732 and 6,277,608, U.S. Patent Application Publications 2001283529, 2001282319 and 20020007051, all of which are incorporated herein by reference. The GATEWAY™ Cloning Technology Instruction Manual which is also supplied by Invitrogen also provides concise directions for routine cloning of any desired RNA into a vector comprising operable plant expression elements.

The recombinant DNA constructs will comprise 5′ and 3′ regulatory elements in addition to the DNA encoding the protein. The 5′ and 3′ elements can be the native DNA associated with the coding DNA or can be endogenous to the coding DNA. Regardless, the 5′ regulatory element for the RNase DNA should be a female tissue promoter element and the 5′ regulatory element for the SLF DNA should be a male tissue promoter element. The recombinant DNA can be stacked with DNA for imparting other traits e.g. herbicide resistance or pest resistance or other trait such as cold germination tolerance, water deficit tolerance and the like.

In practice DNA is introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Methods and compositions for transforming plants by introducing a recombinant DNA construct into a plant genome in the practice of this invention can include any of the well-known and demonstrated methods. Preferred methods of plant transformation are microprojectile bombardment as illustrated in U.S. Pat. Nos. 5,015,580; 5,550,318; 5,538,880; 6,160,208; 6,399,861 and 6,403,865 and Agrobacterium-mediated transformation as illustrated in U.S. Pat. Nos. 5,635,055; 5,824,877; 5,591,616; 5,981,840 and 6,384,301, all of which are incorporated herein by reference. See also U.S. application Ser. No. 09/823,676, incorporated herein by reference, for a description of vectors, transformation methods, and production of transformed Arabidopsis thaliana plants where transcription factors are constitutively expressed by a CaMV35S promoter.

Transformation methods to provide plants with self-incompatibility are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. application Ser. No. 09/757,089, which are incorporated herein by reference.

At least two lines of transgenic plants with allelic S-locus DNA should be produced and propagated simultaneously to allow cross fertilization for the production of hybrid progeny seeds. When the transgenic plants are produced from the same line, inbred seed can be produced. When the transgenic plants are produced from different lines, hybrid seed can be produced.

Alternatively, the self-incompatible, transgenic plant can be propagated by outcrossing the pollen from transgenic plant line to wild type, preferably an isogenic precursor of the transgenic plant line. Using a selectable marker, e.g. glyphosate resistance linked to the S-locus DNA, will facilitate selection of transgenic progeny, e.g. hemizygous transgenic progeny with the self-incompatibility trait.

In addition to direct transformation of a plant with a recombinant DNA construct, transgenic plants can be prepared by crossing a first plant having a self-incompatible recombinant DNA construct with a second plant lacking the construct. For example, recombinant DNA can be introduced into a plant line that is amenable to transformation to produce a transgenic plant which can be crossed with a second plant line to introgress the recombinant DNA into the second plant line.

### EXAMPLE 1

This example illustrates an embodiment of the production of transgenic corn lines with allelic S-locus genes.

A first recombinant DNA construct is prepared with an herbicide marker and portions of allelic genomic DNA from Petunia inflata S-locus expressing RNase and SLF proteins, SEQ ID NO:1 and SEQ ID NO:2, respectively, including 5′ and 3′ regulatory elements native to the S-locus. The construct further comprises a CaMV 35 S promoter operably linked to a CP4 aroA marker gene and a Tr7 3′ element. The construct is inserted into a binary vector system for Agrobacterium-mediated transformation. Transgenic events in corn callus are selected on medium having glyphosate herbicide. Callus of transgenic events are further selected as having a single copy of the first recombinant DNA construct and no oriV origin of replication from the vector. A first transgenic plant is propagated from the single copy transgenic callus. The first transgenic corn plant exhibits gametophytic self-incompatibility.

A second recombinant DNA construct is prepared with an herbicide marker and portions of allelic genomic DNA from Petunia inflata S-locus expressing RNase and SLF proteins, allelic homologs of SEQ ID NO:1 and SEQ ID NO:2, respectively. The construct further comprises a CaMV 35 S promoter operably linked to a CP4 aroA marker gene and a Tr7 3′ element. The construct is inserted into a binary vector system for Agrobacterium-mediated transformation. Transgenic events in corn callus are selected on medium having glyphosate herbicide. Callus of transgenic events are further selected as having a single copy of the second recombinant DNA construct and no oriV origin of replication from the vector. A second transgenic plant is propagated from the single copy transgenic callus. The second transgenic corn plant exhibits gametophytic self-incompatibility.

The first and second transgenic plants are allowed to cross pollinate with each plant producing hybrid seed that has copies of each of the allelic S-locus gene pairs and the glyphosate tolerance DNA.

### EXAMPLE 2

This example illustrates another embodiment of production of transgenic corn lines with allelic S-locus genes. Recombinant DNA constructs are prepared as indicated in Example 1 except that the S-locus DNA is limited essentially to (a) nucleotides 2027-2997 of SEQ ID NO:1 which are linked to an endogenous corn female tissue promoter as 5′ regulatory element and a Tr7 3′ element and (b) nucleotides 208-2337 of SEQ ID NO:2 which are linked to an endogenous corn male tissue promoter as 5′ regulatory element and a tr7 3′ element. Corn lines with allelic S-locus DNA are cross pollinated to produce hybrid seed with two allelic copies of DNA coding for S-locus RNase and two allelic copies of DNA coding for S-locus F-box protein.

### EXAMPLE 3

This example illustrates the production of other transgenic plants with alleleic S-locus genes. The methods of Example 1 are repeated except that corn callus is replaced with callus from dicot plants, soybean, cotton, canola and sunflower. Transgenic soybean plants exhibit gametophytic self-incompatibility and are cross pollinated producing hybrid seed comprising two allelic copies of DNA coding for S-locus RNase and two allelic copies of DNA coding for S-locus F-box protein. Transgenic cotton plants exhibit gametophytic self-incompatibility and are cross pollinated producing hybrid seed comprising two allelic copies of DNA coding for S-locus RNase and two allelic copies of DNA coding for S-locus F-box protein. Transgenic canola plants exhibit gametophytic self-incompatibility and are cross pollinated producing hybrid seed comprising two allelic copies of DNA coding for S-locus RNase and two allelic copies of DNA coding for S-locus F-box protein.

1. A self-incompatible, transgenic line of a naturally inbreeding-capable crop, said transgenic line comprising in its genome a segment of exogenous, allelic DNA from an S-locus of a self-incompatible plant, whereby said transgenic line produces pollen which is incapable of fertilizes said line with said allelic DNA from an S-locus.

2. A self-incompatible, transgenic line of claim 1 wherein said inbreeding-capable crop is selected from the group consisting of corn, rice, wheat, canola, cotton and soybean.

3. A self-incompatible, transgenic line of claim 1 wherein said segment of allelic DNA from an S-locus comprises DNA encoding an allelic S-RNase and a cognate allelic S-locus linked F box protein.

4. A self-incompatible, transgenic line of claim 3 wherein said allelic DNA from an S-locus comprises promoters native to said S-locus.

5. A self-incompatible, transgenic line of claim 3 wherein said allelic DNA from an S-locus comprises promoters endogenous to said crop.

6. A method of producing hybrid plants of an inbreeding-capable crop, said method comprising:
(a) introducing into the genome of a first line of said crop a segment of allelic DNA from an S-locus of a self-incompatible plant to provide a self-incompatible, transgenic line of said crop,
(b) allowing pollen from a second line of said crop without said segment of allelic DNA to fertilize said self-incompatible, transgenic line of said crop.

7. A method of claim 6 wherein said inbreeding-capable crop is selected from the group consisting of corn, rice, wheat, canola, cotton and soybean.

8. A method of claim 6 wherein said segment of allelic DNA from an S-locus comprises DNA encoding an allelic S-RNase and a cognate allelic S-locus linked F box protein.

9. A method of claim 8 wherein said allelic DNA from an S-locus comprises promoters native to said S-locus.

10. A method of claim 8 wherein said allelic DNA from an S-locus comprises promoters endogenous to said crop.

11. A method of claim 6 wherein said second line of said crop is a self-incompatible, transgenic line of said crop with an allelic segment of DNA from an S-locus distinct from the allelic segment of DNA in said first line of said crop.

12. A method of claim 11 wherein said crop is corn and where said method comprises planting adjacent corn plants of self-incompatible, transgenic lines of corn, wherein each of said lines has in its genome a segment of exogenous, allelic DNA from an S-locus of a self-incompatible plant, wherein each line has a different allele of said allelic DNA.

13. A method of claim 11 wherein said crop is selected from the group consisting of corn, rice, wheat, canola, soybean and cotton.

14. A hybrid seed comprising two allelic copies of DNA coding for S-locus RNase and two allelic copies of DNA coding for S-locus F-box protein.


SEQUENCE LISTING (2 sequences)

SEQ ID NO: 1 - DNA, 3850 bp, Petunia inflata
  misc_feature: S-locus RNase gene
tgccatgggtaatcgaggacaggggactgctggtagaggccagtcgagagtttttgcacttactaggcaagatgctcaggcctcaaatgctgtggttacaggtattctttctgtttgctcacttgatgcacttgcattgtttgatccaggatctacccactcctatgtgtcctcattctttgctttgaggtttgatagacaaccagaaatattgaatgatccttttctagttgctaatcctgtgggagattctttacttgttgaatatgtgtatcgtgcttgtcagattcgagttgagggtagggataccttagctgatttaattgtactggatatggttgattttgacgtgttgatgggtatggattggttatcttcttgccatgctacccttgattgtcatgcgaaggtagtgaagttcgagatgcctaatgaacctagttttattctgaaaggggatcaaattctagaaatgtgtaatgtcatatcatttgtaaaagctcagcagttagtgaagaaaggttgttgggcattcttagctatggtgagagacacaagtgatgtgactcctagcttggaaatggttccagtggtgcaagagttcactgatgtattaccgggactatttagagtcgctccatttagtcatttgtcagtcattcttttagtcagttggattatttatttataagctagtcttgagagtatttggtctttatgaacttatcacaatcctgttagacgttctcgtagtttgtgggagataaatgtggtatttttattgttaatgcgtaagaatattggttgttttgctgggttttctgtttgtttaaagcaggtaaaattttggataggcctaaaaacaggggaaactctgcccgattttctgtagaatattgataaggcttgtttagggacttagttcctaagcgccggtcacggtctcaaattgggtcgtgacaatattatattttttatctcaatttatttgttttaatcatatatttccttatacgttttatgtaggtctaggatcattaataaatacttatgcaccaaatgaaagagcctgataaatgcttgttatgtttttttacgaggacgacaagttaatcactaatttttatttaattgttttcggctagaaaaaattattttgctcctgataataattttatttttaaaattttcaaatatataactacgtagtaatatttgtgaaaccaaaatatgcatgtaattgcattttaataacgctatggcgaaaaaatagatctctataattatggtactagtagtttgccaaattcaatatcatagtgttcctccaaacaccatttaagattaaatagttgttgaattcatttaataaactgttatagaccaaagagaagagctcgacccaaaagactagtctaataggtgggagagcccatttagtctatcaatgtgggagaggtgctcacaataatcgtccccgcttgaggaccaacaacctcgttggtcacggttgcccagttggtcacggctggcacccctcttaggttcaggccaccactcctggcataagccaccaatccgagtatcccgttggtcaatggctatccagttggtcacggctggcacccctcttgggtccaggccatcactcctagttgtggcccccaagggtggattctcaatgaaagcaccaatgttatagaccaataggagagcccaacccaaactagtctagtaggtgggagagtccttttaatatatatactcattagcatctctctttttttatcaatgtggaagaggtgctcaccataataacacttgtaacttcaatttatgggtataataaatcataaaaagatcacccatcgctttttccactccacctgtgatataaggtaacttaaaaatttaactagcagttcagtggtttggttgttcctaattataaaacgtcccaccgatagaaaagcgattatatatagccccggaagaaatgatgagaatatattcaacgtactagaatatgtttagattacagctcttgtcagctcttttcattttgcttttctctctttctcccgttagtgccaattttgactacttccaactcgttttaacatggccagcatccttttgctaccctaagaatttttgcaagagaaaatcgaacaacttcacaattcacgggctttggcccgaaaataagcattttcgtctagagttctgcactggcgacaagtattcgcgttttaaagaagtacaaaattgtttccttaagcgtttctaaattttatgtttttctctcaatgttcctagtcctttgtggtcattttaattgaatagttttttactaacttttacaggataatataatcaatgttctggagcgccactggattcaaatgaggtttgacgaaaaatacgccagcactaaacaacctctctgggagcacgagtacaataggcatggaatttgctgcaaaaatctctacgatcaggaggcttattttttactagccatacgcttaaaagataaacttgatcttttgacaactctcagaactcatggaattactccgggaacaaagcatacatttggtgaaatccaaaaagccataaagacggtcactaataataaagatcctgacctcaagtgcgtcgaaaatatcaaaggagtaaaggaactaaatgaaataggcatatgttttaacccagcggcagatagttttcatgattgtcgtcacagcaaaacatgcgatgaaacagacagcacgcagactctgtttcggagatgaatgattatcatttctttttttctgttcattcttactaagaaaaagacaagtcgaggtcatctaagaagaatatagtaccgatagacctctttgtggaacaactgaaataaatttataatcgatttacgaaatcattttgtcttaaacttagtggcgtttatgtagtgctgtcaatatgggccggcccgacccagcccgacccagcccacgtggactttgcagtttgccgggctgggtcgggccggcccacaatttgtgtgggcctttaaatcattagtccagcccagtcctatgtgggctgcaggccgggtcgggccagcccatcaatattaatttttaattttttttttaaataatgagttttgattttgaatttgataacctttaaaactagaaatatacaagtattactattacttttagttaaaaaattgtaaaaactttgatcttaacaggtacttgatttggactttagtcatttcaagtttcaactttcaatttccattaatcaaaagttttagatgcaacttcagataatacaagatagtaagaataaaaccacaaatttaacataactgatattttccatcaacctgcattctaaacaacattaaaactaaaatataaataaaaagtattataaagatttctagtatgtaagtacttaaaaattgcaaaaaaaaaaatattgaaaatggaaggctacatttcattcacaatagcttatccaattccaccacatcgtcatcatgagcatctggatcttcttgcaataaagttgtcataatatcttcattttcatctatatctacaattcaatgtgagttagttaaacattaataaatatctaaattttaagaactaataaaatttactttaaataaaaaattataccagtttgcttttgggtaaagccatgcaaccaatttcgggtagtaacaagtttttggacgttatcatgatggatgcaacttctatacttagtgagaacacgaccaccaatgctgaatgccgattcagatgctacagtagtaataggaatactaagtaca

SEQ ID NO: 2 - DNA, 4300 bp, Petunia inflata
  misc_feature: S-locus F-box gene
taaggatagagtggttaaatctttgctttctatgatagcacaagctttgatctcccaagaatctggaagaaccctagttaaaatcttttctactctatcttccacagttaaaacccttcccaaagagataagttcagaagcaatggcattaaatctagtatacatttcagagattgactcttcttctttcattttaaagctttcatattcagcataaagaagtgtacctcttgacctttttacctgacttgttccttcgtgtacaacttgtagagcatcccaaatctcttttgcagtggcataccctctgattctagtgcatttgtcaggactaagtccaccaaccagccacttccttgcctttgcattcttttcccattgctttatgtcttcagcattgcattccttcctggttttgggaaaatcatttccctcggcatccttcttcattgggatcatgggaccttcagttactatgtcccacaattcatagtcctctgcttgaaggtgatcacgcatcctttctttccaccatgaatagtatttcccattgaagtaaggagctctaaccattgactgaccctcactgttctctcgtgatgtactcatctaatcctaggtgttagccttttcaaggataacctgctctgataccaattgttatttcccacgattacaccactttactgttttaaggaccagttccttaaaatgtgttcctttactctacgatatgcggaattaaaactgtaagtaattgaacaccagatattagcgtggaaaaaccccttgactcgggatcaaaaaaccacgacctacttctcaagtaggatttcaacttcactatgtaaaaagacccacttggctacaagactcactcttactaacctaggactaactctaatcctttagtgatctttctcacgatcagtgaagttgtgtcaagtccacgacttgaaccagaaagtacaactcgatttttgcttactacaattgaactaagacttgaacttttacatgactcaatcttaagcaacaggttctaacttaattctactgtagctaactgattacacaaataagctaataactcttttaagcttttgctcgatgctctctgtatttgtgcttagcttctacggctgaaatgctccacattttatagctccagcagccaagaagtcttcaataattagaactccaatgctcccttgagtttgagtcctaattggattcttctctttagaatccggatcttctatccttatccttgtgtttatgcaatcttttcatatccgtttagacctgcataattatcaagataaacattagttttaggataattaataatatctcaacttaaacgattttgcatgaattatttatttagctgcagtgattcaaaggaacaggtcctttgaatctgttcctcagtcatcagtcaaaactccaagcttatccaagctaacactaataaataaaaagtcagttcaagtaccacaatgagaatttatgttaacttttgcttgtgtattcgaccttttaattactgaagtgggtttagaattataatattgataatgttagataaaatgtaaaaagtaaatatactactaaccaaattcatgtatccatttaataatttgggaagtaaaatatgataatgtcttacattgtgtaaggtaagtagaaaattgacaagaattctttatattttgtcccatttggaatatcgccccattctttagaaacttgggtaaaaatcagagtcatgtaatacttagtttcaaagagattgatggtaattttacaaaggtaaaaaaatgaaagttaatatagttaacgaaccattggtagttattatagctccttaaaagaaaattgtttcgatagaaacaatcacaaccacaaaatcagacgttcgaagttcaaagagatctaatcttctgtctaaactttttgatccagttgaatgagatggaaactcctcttctgataaagctgtaactacacagagaatcactattgttaaaggtgcaatccaaagtgcattcttaagaataaaggatggcgaatggtattttaaagaaattgcccgaagatttggtgtttcttatactattaacatttccagtgaaatctcttatgcgattcaaatgtatctctaaagcttggtccattctcatacaatcaactactttcataaaccgtcatatcaatcgcaaaacaaacacaaaagatgaattcattctcttcaagcgtgccatcaaagatgatgaagaagaatttataaatatcttgtcttttttttctggtcatgttgatgttcttaaccctctttttccagatatggatgtgtcatacatgacctccaaatgcgattgcacttttaatccactcatcggtccttgtgatggtttgattgctttgacagataccataatcaccatagtacttaatccggctaccagaaacttcagagtgctcccagctagcccttttggttgtccaaaaggttaccatcgttctgttgaaggggttgggtttggcttggataccatttcaaattactataaggttgttaggatttctgaagtttattgtgaagaagctgatggttatcctggtcctaaagatagtaaaattgatgtttgtgatttgagtactgattcttggagagaattggaccatgtacagttgccatcgatttattgggtgccttgtgctggaatgctttacaaggaaatggttcactggtttgcaactacagacatgtcgatggttattctttgttttgacatgagtactgagatgtttcatgatatgaaaatgcctgatacttgtagtaggattacgcacgagctgtattatggccttgtaatcttatgtgagtctttcacattgattggttactccaacccaatcagttctactgatccagcacacgataaaatgcacatttgggtgatgatggagtacggtgtcagcgagtcttggattatgaaatacactattagacctctgtctattgaatcccctttagctgtttggaagaatcatatattgcttcttcaatgtagaagtggacttctaatttcctatgatcttaattccggtgaagcaaaggaattgaacttacatggttttccagacagtttgagtgttaaagtttacaaggaatgcttaacttcaattccaaaagggagcgagtacagtacaaaagtacaaaaattttagaaacggtgcaactctttgtgacgaatttgcacataaatttcagcagctttttcttccaaagattctaattgttgtaataggcagcttgtttatatatgctacttagtaaaaaagctttaccatgtcaatatgtttgttttgtttttaccaaaaatagttttagtaccatattctttcttaatatttcatctaagaatcatatgacgatgcaattttacatatttttctttaatgtgccatgagtaactccatcctttcttttttaacattatattgtttttattaattggaaatgatcaataaatcattagtttttctttcttctttgagctgtaaaaacaattggtaatgctctaaacactacaagaaaacagctaaatcctagcagttcttttgcagcgatttagataaactgccgggaaatatcattttgcagcggttctattagccgcataagtaatgagttgtattacacttgagacctgttgctcctaaacgcacacgcaagtatacgtggtcgacaagtaataaagtgataaaaatgaatatcgttgccacgaggactattgttcagagtgaatcctagcaaacaatttcgtaaaaactattcaggaatctttttcaagtgtgattgagatttttactaactaaagaaaacagtaattaaaaacaagcaactgagatcaagtaatagagcaaattctaggttgttatcaataaaagagaaacggtccagggtcagagctacaaacgatccagttgagttttccagttgattctacttcttgattcgtgaaaacctgtcgattcttcacgagtctatgcaagctactctaacgcctatatttctatggaattaaatcacatgcatgcattaaagaactctgtatttagctaagcaaggcgaataggtatatgtttatcctacccgcaaatcgattccctgatgctcaggttcaaaatcaagccctattctttcttatttgcattcacaggttccctctgtcgagttcaatttgtaaaccgtagatagtgttcaattatcaataaccaaacaattagg